(19) United States
(12) Patent Application Publication
(10) Pub. No.: US 2026/0092916 A1
(43) Pub. Date: Apr. 2, 2026

(54) TEST STRIPS AND METHOD FOR EXTENDING SAMPLE FLOW PATH IN LATERAL FLOW ASSAYS

(71) Applicant: Inito Health Inc., Houston, TX (US)

(72) Inventors: Varun AKUR VENKATESAN, Bangalore (IN); Siddharth PATTNAIK, Bangalore (IN); Dipankar DAS, Bangalore (IN)

(21) Appl. No.: 19/411,420

(22) Filed: Dec. 8, 2025

Related U.S. Application Data
(63) Continuation-in-part of application No. 17/620,761, now Pat. No. 12,517,116.

(30) Foreign Application Priority Data
Jun. 20, 2019 (IN) 201911024493

(51) Int. Cl. G01N 33/543

(52) U.S. Cl. CPC G01N 33/54393; G01N 33/54388

(57) ABSTRACT

The present disclosure provides a method of extending the path of a biological sample flow in a lateral flow assay on a test strip including a membrane. The method includes arranging the membrane in a helical shape in a test strip. The helical shape extends the flow path increasing interaction time between the biological sample and binding reagents on the membrane as the sample flows through multiple turns of the helix. Further, a lateral flow assay on the test strip including a membrane disposed on a surface of the test strip in a helical shape is provided. The helical shape extends a path of a biological sample flow through the membrane.

### CROSS-REFERENCE TO RELATED APPLICATIONS

This application is a continuation-in-part of U.S. patent application Ser. No. 17/620,761, filed 2021 Dec. 20, which published as U.S. Patent Application Publication No.2022/0341919 A1 on Oct 27, 2022, the entire disclosure of which is incorporated herein by reference.

### FIELD OF INVENTION

The present disclosure relates to lateral flow assays, and more particularly to test strips and method for extending sample flow path in lateral flow assays.

### BACKGROUND

Lateral flow assays are widely used for detecting analytes in biological samples such as urine, blood, saliva, and other bodily fluids. These assays rely on the flow of a sample through a membrane where binding reagents are immobilized to capture target analytes. The sensitivity and accuracy of lateral flow assays depend significantly on the interaction time between the sample and the binding reagents on the membrane. Insufficient interaction time may result in incomplete binding, leading to reduced sensitivity and potentially false negative results, particularly for samples with low analyte concentrations.

Conventional lateral flow assays typically employ linear membrane configurations where the sample flows along a straight path from the sample application zone to the detection zone. While this design offers simplicity and ease of manufacture, the limited flow path length restricts the interaction time between the sample and binding reagents. Increasing the length of a linear membrane to extend interaction time results in larger device footprints, which is impractical for point-of-care applications where compact, portable devices are preferred.

There remains a need for lateral flow assay configurations that provide extended interaction time between samples and binding reagents while maintaining a compact form factor suitable for point-of-care testing applications.

### SUMMARY OF INVENTION

In some aspects of the present disclosure, a method of extending the path of a biological sample flow in a lateral flow assay on a test strip including a membrane is provided, said method including arranging the membrane in a helical shape in the test strip.

In some aspects, the test strip further includes a sample pad and a detection pad disposed on the surface of the test strip.

In some aspects, the helical membrane is disposed completely or partially on the inner or outer surface of the test strip.

In some aspects, the biological sample flows along the helix of the helical membrane.

In some aspects, the membrane is configured to facilitate biological sample flow in a direction substantially perpendicular to a central axis of the helical shape.

In some aspects, the test strip further includes the detection pad disposed on the test strip, such that the biological sample flows into the detection pad in a direction substantially perpendicular to a central axis of the helical membrane.

In some aspects, the extended path increases interaction time between the biological sample and binding reagents on the membrane.

In some aspects, the biological sample flows through multiple turns of the helix sequentially.

In some aspects, the biological sample flows in a direction substantially perpendicular to a central axis of the membrane.

In some aspects, the method further includes detecting an analyte in the biological sample, such that the increased interaction time enhances binding between the analyte and binding reagents.

In some aspects, the biological sample flows radially outward from the central axis.

In some aspects, the biological sample flows radially inward toward the central axis.

In some aspects, detecting the analyte includes visual observation, fluorescence, chemiluminescence, or colorimetric readout.

In some aspects, the helical configuration reduces flow velocity, thereby increasing residence time of the biological sample within the membrane.

In some aspects of the present disclosure, a lateral flow assay on the test strip including the membrane disposed the on a surface of the test strip in the helical shape is provided, such that the helical shape extends a path of a biological sample flow through the membrane.

In some aspects, the helical shape increases interaction time between the biological sample and binding reagents on the membrane.

In some aspects, the lateral flow assay further includes detection pad disposed on the surface of the test strip.

In some aspects, the membrane is configured to facilitate biological sample flow in the direction substantially perpendicular to a central axis of the helical shape.

In some aspects, the biological sample is selected from urine, semen, sperm, blood, and saliva.

In some aspects, the biological sample flows continuously from an inner turn toward an outer turn of the helical membrane.

In some aspects, the helical shape includes multiple turns arranged along a longitudinal axis of the test strip.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 is an isometric view of a test strip assembly 101 of the device for analysis of fluids, according to an embodiment herein;

FIG. 2 is a top view of the test strip assembly 101, according to an embodiment herein;

FIG. 3 is an isometric view of the test strip assembly 101 showing a minimum of three detection labels, according to an embodiment herein;

FIG. 4 is a top view of the device for the analysis of fluids showing two test strips assemblies of FIG. 1-3, placed parallel to each other, according to an embodiment herein;

FIG. 5 is a top view of the device of FIG. 4 inserted into a housing, according to an embodiment herein;

FIG. 6 is a side view of the device of FIG. 4, according to an embodiment herein; and

FIG. 7 shows the flow of the analyte sample fluid through capillary action in the test strop of FIG. 1, according to an embodiment herein.

FIG. 8 illustrates a lateral flow assay test strip having a single-start helical configuration disposed on a test strip, in accordance with some aspects of the present disclosure, according to an embodiment herein.

FIG. 8A illustrates a variation of FIG. 8, showing a lateral flow assay test strip having a variable-pitch helical configuration, in accordance with some aspects of the present disclosure, according to an embodiment herein.

FIG. 8B illustrates a variation of FIG. 8, showing a lateral flow assay test strip having an interlaced dual-helix configuration, in accordance with some aspects of the present disclosure, according to an embodiment herein.

FIG. 8C illustrates a variation of FIG. 8, showing a lateral flow assay test strip having a multi-start helical configuration with parallel helix channels, in accordance with some aspects of the present disclosure, according to an embodiment herein.

FIG. 9 illustrates a lateral flow assay test strip having nested coaxial helices configuration, in accordance with some aspects of the present disclosure, according to an embodiment herein.

FIG. 9A illustrates a variation of FIG. 9, showing a lateral flow assay test strip having a conical helical configuration, in accordance with some aspects of the present disclosure, according to an embodiment herein.

FIG. 9B illustrates a variation of FIG. 9, showing a lateral flow assay test strip having a folded helical configuration, in accordance with some aspects of the present disclosure, according to an embodiment herein.

FIG. 9C illustrates a variation of FIG. 9, showing a lateral flow assay test strip having a toroidal helical configuration, in accordance with some aspects of the present disclosure, according to an embodiment herein.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Conventional linear lateral flow assays are limited by short interaction times between samples and binding reagents, as membrane length directly constrains flow path, reducing sensitivity for low-concentration analytes. Increasing membrane length in a linear format enlarges device size, which is impractical for portable point-of-care applications. The present disclosure overcomes this by arranging the membrane in a helical configuration, converting the flow path from two-dimensional linear to three-dimensional winding. This design decouples flow path length from test strip footprint, enabling significantly longer interaction times within compact dimensions. The sample travels through multiple helical turns, maintaining continuous reagent interaction, improving binding efficiency, signal generation, and detection sensitivity without sacrificing portability.

The term “bodily fluid” or “bodily fluid sample” or “biological sample” refers to the fluids in or on a human or animal body. The examples include sweat, urine, blood, blood serum, semen, breast milk, saliva, blood plasma, tears, mucus, cerebrospinal fluids, saliva, amniotic fluid, vaginal lubrication fluids, pus, lymph, bile, synovial fluid, aqueous humour, phlegm, gastric acid, pre-ejaculate, colostrum and other such fluids related to animals or humans. The bodily fluid may also include the bodily matter that has been liquified by mixing in a solvent such as water. The two phrases “bodily fluid”, “bodily fluid sample”, “biological sample” and “sample” are used interchangeably across the present specification.

The term “analyte” or “one or more analytes” refers to hormones, ions, proteins, lipids, sugar, oxygen, antibodies, enzymes, carbohydrates, virus, bacteria or any other foreign particles, metabolites that may be detected and analysed, qualitatively or quantitatively, to determine the state of health or general well-being of an animal or a human

The following description sets forth exemplary aspects of the present disclosure. It should be recognized, however, that such description is not intended as a limitation on the scope of the present disclosure. Rather, the description also encompasses combinations and modifications to those exemplary aspects described herein.

The terms ‘user,’ ‘patient,’ ‘body,’ and ‘subject’ may refer to similar meaning/interpretation and may be interchangeably used throughout the specification.

The term “test device” and “device” are interchangeably used across the context.

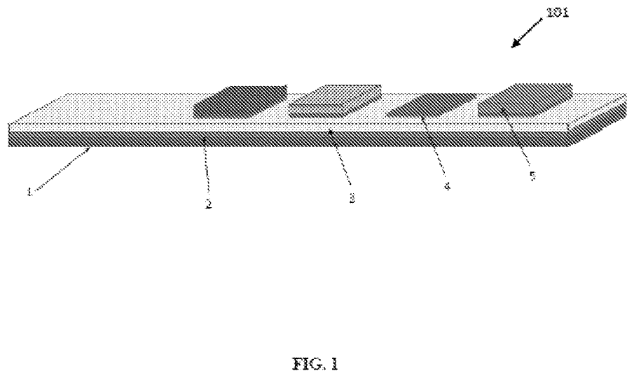

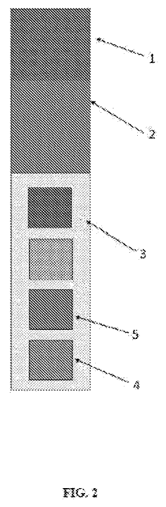

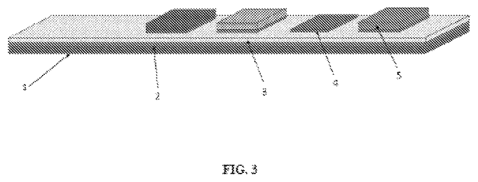

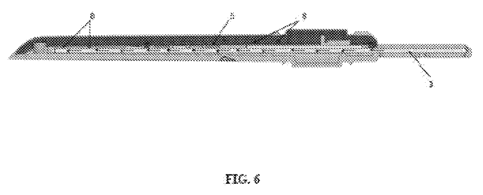

Referring now to the drawings, and more particularly to FIGS. 1 through 7 <incorporated from U.S. Patent Application Publication No. 2022/0341919A1 entirety>, where similar reference characters denote corresponding features consistently throughout the figures, there are shown preferred embodiments.

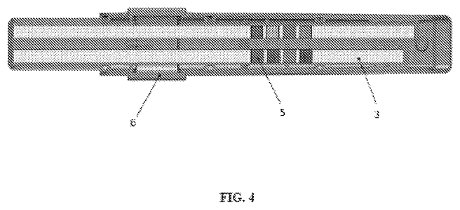

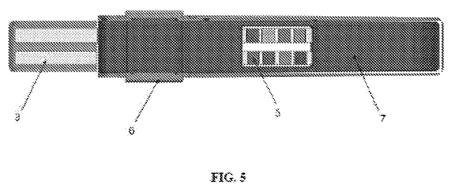

FIG. 1-3 illustrate different views of a test strip assembly 101 of the device for analysis of fluids, according to an embodiment herein. The test strip assembly 101 includes a basal layer 1, a first adhesive layer 2, a porous membrane 3, a second adhesive layer 4, and a plurality of detection labels 5. In an embodiment, the first adhesive layer 2 is entirely present over the basal layer 1. In an embodiment, the porous membrane 3 is present over the first adhesive layer 2. The number of detection labels 5 are connected with the porous membrane 3 through the second adhesive layer 4. In a preferred embodiment, the porous membrane 3 is smaller or equal in dimension to the basal layer 1. In an embodiment, the detection labels 5 are smaller in dimension compared to the porous membrane 3 and vary in number on one test strip 101. The entire test strip assembly 101 is placed into a customized cassette/housing 6, as shown in FIG. 4-6, which is greater than or equal in length to the test strips assembly 101. The cassette/housing 6 has an opening. The opening is covered by a removable cap. The opening is designed to expose the zone containing the detection labels 5 on the test strip assembly 101 in order to be read by naked eyes or by a reader. In an embodiment, the housing 6 can house a number of test strips 101 to run several kind of tests parallelly and/or simultaneously using different bodily fluids at the same time. In an embodiment, the housing 6 houses the number of test strip assemblies 101 such that each of the test strip assembly is parallel to each other. FIG. 4 and FIG. 5 illustrates a housing 6 having two test strip assemblies 101 lying parallel to each other, within the housing. The housing, thus, can be adapted to include as many test strip assemblies 101 as possible to have multiple detection and/or diagnosis using different bodily fluids simultaneously at the same time.

According to an embodiment of the present invention, the basal layer 1 of the test strip assembly 101 is made up of resins, metal foils, or glass. The resins are selected from the group consisting of polyvinyl alcohol, polystyrene, polyvinyl chloride, polyester or a polyamide. The adhesive layers between the basal layer/sheet 1 and porous membrane 3, and between the porous membrane 3 and detection labels 5 is made up of fusion of adhesives like polyethylene or polyvinyl alcohol. The first adhesive layer 2 covers the entire top surface of the basal layer 1. The adhesive layer 2 needs to be hardened so that the abutting of basal sheet 1 and porous membrane 3 is firm. The second adhesive layer 4 covers the bottom surface of the detection label 5 entirely or enough firmly affix the detection label to the porous membrane 3. The material used as adhesive can be the same for both the adhesive layers.

The porous membrane 3 is made up of cellulose based polymers such as nitrocellulose membrane and nonwoven cellulose fibre membranes. Alternatively, blends of natural and synthetic polymers such as CytoSep is also used depending on the application. Glass fibres are also used as a material for the porous membrane layer. Typically, the thickness of the porous membrane should be between 15-200 microns and 40-80% of the surface should be porous. The membrane can be varied according to the type of metabolite to be analysed, within the given limits.

In a preferred embodiment, the porous membrane in the strip 101 is made up of several material. In a preferred embodiment, the porous membrane is made up of are either woven polymers, cellulose polymers, glass fibre polymers or mixed polymers that are a mixture of natural and synthetic polymers. The examples of woven polymers include cotton and nylon. The examples of cellulose polymer membranes include nitrocellulose membranes. The examples of mixed polymers include membrane materials such as CytoSep and Vivid Plasma Membrane. The pore sizes for the membranes typically range from 8-15 microns for all samples with very low viscosity such as urine or water. In an embodiment, the low viscosity fluids have viscosity less than 0.002 Pa-s. However, while working with highly viscous fluids such as whole blood, membranes with low pore size ranging from 0.1-5 microns are generally used. In an embodiment, the high viscosity fluids have viscosity higher than 0.002 Pa-s.

The detection labels 5 preferably includes an absorbent carrier such as filter paper impregnated with reagents that change colour upon chemical reaction with the metabolite. In an embodiment, a mix of detection reagents with neutral solid materials can also be used as detection labels. The reagents are immobilised on detection pads or labels 5 in a manner that avoids cross-contamination of reagents i.e. immobilized reagent doesn't flow along with the analyte in a bodily fluid. In a preferred embodiment, the absorption properties of all the detection pads or labels in the assay must be the same in order to maintain a uniform flow rate from start to end of the assay. In a preferred embodiment, the thickness of all detection pads or labels 5 is kept same and the distance between them is maintained in order to enable uniform availability of the analyte, in a bodily fluid, at different detection pads or labels 5.

The device and the test strip 101 assembly is required to be dipped partially into the bodily fluids sample meant for analysis such that only the tip of the device is required to be dipped.

In a preferred embodiment, the direction of flow of the liquid analyte or bodily fluid is lateral i.e. along the membrane 3, and the flow of the same analyte, after contacting with detection pads/labels 5, is vertical in the detection pads or labels 5.

The labels for bodily fluids may be for detecting glucose, ketones, uric acid, bilirubin, urobilinogen, pH and specific gravity. In an embodiment, a plurality of detection labels 5 may be arranged on the test strip 101. In an embodiment, the test strip 101 includes label for testing pH, which includes reagents such as methyl red, bromothymol blue and methanol or a mix of them. In another embodiment, the test strip 101 includes label for testing presence or absence of protein, which includes reagents such as sodium Sodium citrate, Citric acid, lauroylsarcosine, water, Magnesium sulfate, Tetrabromophenolphthalein ethyl ester and methanol or a mix of them. In yet another embodiment, the test strip 101 includes a label 105 for detection of urobilinogen in urine and the label includes reagents such as 4-cyclohexylaminobenzaldehyde, Oxalic acid, Methanol or a mix of them. Similarly, the label 105 for detecting glucose includes O-tolidine, Peroxidase, Glucose oxidase, Tartrazine, Ethanol (44%) or a mix of them. In still another embodiment, the test strip 101 includes a label 5 for detection of hydrogen peroxide via reagents that include Polyvinyl propionate dispersion, Phosphate buffer, Sodium alginate, Sodium lauryl sulfate, O-tolidine, Peroxidase and Methanol or a mix of them. Similarly, the presence of nitrates may be determined by the test strip 101 through the label 5 incorporating reagents that include sulphanilamide, a-naphthylamine, tartaric acid and methanol or a mix thereof.

The layer of porous cellulose polymer membrane in between the backing sheet and the detection labels allow the liquid analyte to travel from the point of contact with strip to the detection labels by capillary action. This ensures a controlled flow of the analyte to the detection labels which makes the analysis easier and more accurate. Furthermore, due to this porous membrane enabled capillary action, only the tip of the strip is required to be dipped in an analyte solution.

The plastic cassette/housing 6 and top cover 7 are made up of plastic materials such as polycarbonate plastics, acrylonitrile butadiene styrene, high density polyethylene, polystyrene and polypropylene. A combination of two or more plastics can also be used. Preferably, the same material is used for the plastic cassette and the cap.

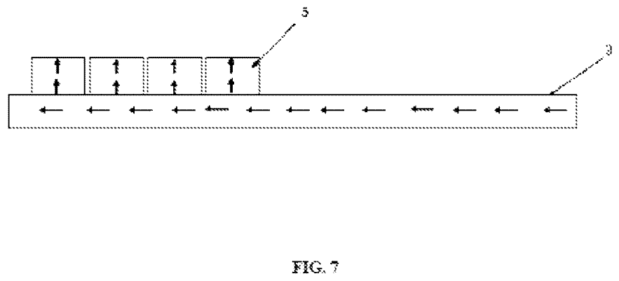

The assembly of the test strip 101 circumvents the need for dipping the entire strip into the liquid analyte. In addition, placing the test strip in a cassette aids easy handling. The user may hold the housing from one end and the open end of the test strip can be dipped into the analyte solution. Once the analyte reaches the detection layer by capillary action, it can access the detection layer preponderantly from beneath, wetting the detection label which gives a quick and readable colour reaction. FIG. 7 shows the flow of the analyte sample fluid through capillary action, according to an embodiment of the present invention. The plurality of openings 8 act as pinch points in the cassette for flow control of analyte sample. Thus, the test strip provides an easy and a more controlled way of detecting metabolites and metals in liquid analytes, which signifies greater consistency in the test results from one test to another.

In an embodiment, the device is adapted to be read by an optical device such that the labels 5 can be read using smartphone's or any handheld device's camera and light source to detect presence or absence as well as quantity of any constituent of any bodily fluid. In an embodiment, device is inserted into an optical device having a transparent optic defining an optical volume, a transparent optic having a first main face adapted for positioning the test strip assemble 101 for labels 5 to be imaged. The transparent optic is adapted to admit into the optical volume a light emitted by the light source for illuminating the labels 5 and wherein the transparent optic is adapted to admit the light having interacted with the labels 5, into the optical volume and turn the light inside the optical volume such that the light is internally reflected within the optical volume and exit the optical volume to be incident onto the camera. Alternatively, the device of the present embodiment can be inserted into a stand-alone or specialised optical readers or devices meant to detect presence or absence of an analyte in a bodily fluid.

In an embodiment, a method for detection of an analyte in the bodily fluid sample using a strip that is only required to be dipped partially into the bodily fluid sample is provided. The method includes dipping the device of FIG. 4, having the housing that carries or encloses the test strip assembly of FIG. 1 into the bodily fluid sample such that bodily fluid sample travels in a lateral direction in the porous membrane of the test strip assembly and the detection label, placed on the porous membrane, receives the bodily fluid such that in the detection label flow of the bodily fluid sample is in vertical direction. This is followed by subjecting the device into an adapter that can be adapted to or attached to a handheld camera device such as a smartphone, such that the detection labels are read by the camera, to detect the presence or absence of any analyte in the bodily fluid sample and convey the results of the test to a cloud server or locally.

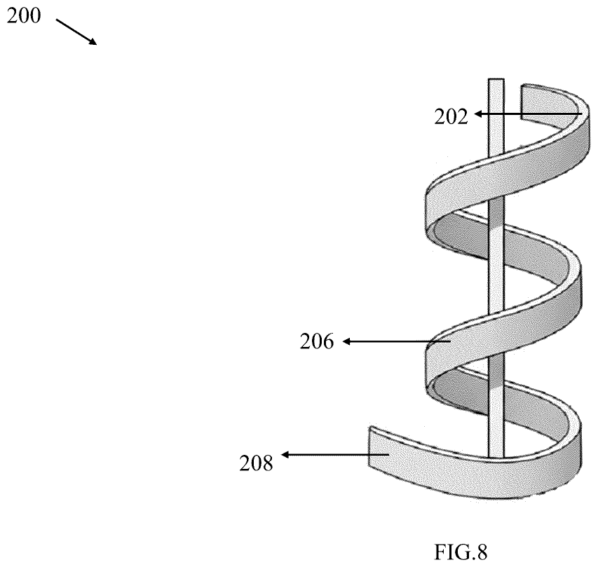

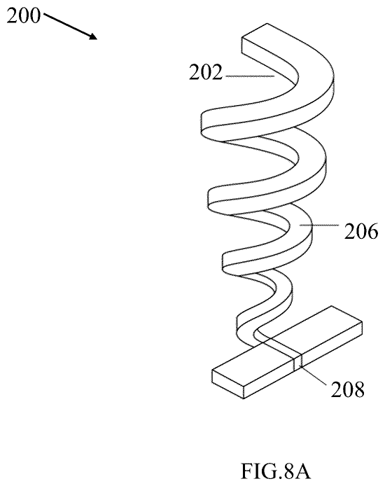

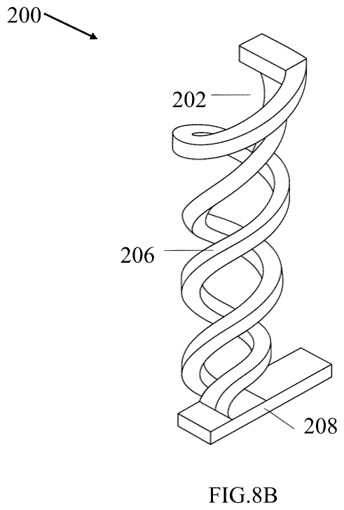

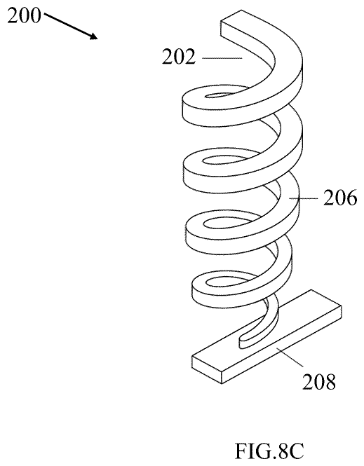

Referring to FIG. 8, a lateral flow assay test strip 200 having a helical configuration is illustrated. In this aspect, a membrane is disposed on a surface of the test strip 200 in a helical shape, whereby the helical shape extends the path of biological sample flow through the membrane.

In lateral flow assays, a biological sample migrates through a membrane where binding reagents are immobilized to capture target analytes. The efficiency of this binding and the sensitivity of detection are influenced by the interaction time between the sample and the reagents. This interaction time is largely determined by the length of the flow path. Conventional lateral flow devices employ a linear membrane configuration, where the flow path length is constrained by the physical dimensions of the device. Extending the path in a linear format requires proportionally increasing the device size, which is impractical for compact, portable point-of-care applications.

The present method introduces a geometric solution that extends the flow path length without enlarging the device footprint. By arranging the membrane in a helical configuration, the flow path is transformed from a two-dimensional linear strip into a three-dimensional winding path. This design decouples flow path length from device size, enabling a significantly longer path within the same or smaller spatial dimensions. For example, a helical membrane with multiple turns can provide a flow path several times longer than a straight strip of equivalent width, while occupying a footprint determined primarily by the helix diameter rather than its total length. This approach efficiently utilizes three-dimensional space and represents a fundamental shift in lateral flow assay design.

The helical configuration offers multiple performance benefits. First, the extended flow path increases the residence time of the biological sample within the membrane, allowing more extensive interaction between analytes and immobilized reagents, which may enhance capture efficiency and signal generation. Second, the geometry inherently moderates flow velocity through capillary effects and increased surface area, further improving interaction time. The sample may flow radially inward or outward relative to the central axis or along the helical turns, ensuring sequential contact with binding zones. Collectively, these features enable improved assay sensitivity while maintaining the compactness and portability essential for point-of-care diagnostics.

In FIG. 8, lateral flow assay having the test strip 200 having a helical configuration is illustrated. In this aspect, a membrane is disposed on a surface of the test strip 200 in a helical shape. In some aspects, the test strip 200 may include the sample pad 202, a detection pad 206, and an absorption pad 208 disposed on the surface of the test strip 200. The sample pad 202 may be configured to receive the biological sample, whereby capillary flow through the membrane may be initiated. Various types of biological samples may be received by the sample pad 202 including, but not limited to, urine, semen, sperm, blood, saliva, or other bodily fluids.

In some aspects, the test strip 200 may further include the sample pad 202 where the biological sample is initially applied. The sample pad 202 may be configured to receive various types of biological samples including, but not limited to, urine, semen, sperm, blood, saliva, or other bodily fluids. Upon application, the biological sample begins to flow through the membrane by capillary action. Aspects of the present disclosure are intended to include or otherwise cover any type of biological sample, without deviating from the scope of the present disclosure.

In some aspects, the test strip 200 may further include a conjugate pad <not shown> positioned along the flow path. The conjugate pad may contain conjugate reagents that are released upon contact with the biological sample. These conjugate reagents may include labeled antibodies, antigens, or other binding molecules that interact with target analytes in the biological sample. As the biological sample flows through the helical membrane, the conjugate reagents mix with the biological sample and travel together through the extended helical path. Aspects of the present disclosure are intended to include or otherwise cover any type of reagents, without deviating from the scope of the present disclosure.

In some aspects, the test strip 200 may further include the detection pad 206 disposed on the test strip 200. The detection pad 206 may be positioned such that the biological sample flows into the detection pad 206 after traversing the helical membrane. In some configurations, the biological sample flows into the detection pad 206 in a direction substantially perpendicular to the central axis of the helical membrane. The detection pad 206 may be in the form of a zone, line, pad, or area, and is not limited to these forms. Aspects of the present disclosure are intended to include or otherwise cover any type of detection pad, without deviating from the scope of the present disclosure. In some aspects, the detection pad 206 includes a test line where capture antibodies or other binding reagents are immobilized to capture specific analytes. In some aspects, the detection pad 206 may also include a control line to verify proper flow and assay function. Aspects of the present disclosure are intended to include or otherwise cover any number of detection pads, without deviating from the scope of the present disclosure.

In some aspects, the test strip 200 may further include the absorption pad 208 positioned downstream of the detection pad 206. The absorption pad 208 may be configured to receive and absorb excess biological sample after the biological sample has passed through the detection pad 206, thereby maintaining continuous capillary flow throughout the assay and preventing backflow.

In some aspects, the helical membrane may be disposed completely on the surface of the test strip 200, with the entire membrane arranged in the helical configuration. In some aspects, the helical membrane may be disposed partially on the surface of the test strip 200, with only a portion of the membrane arranged helically while other portions may have different configurations. The helical membrane may be disposed on an outer surface of the test strip 200, where the helical membrane may be readily accessible for observation and detection. Alternatively, in some aspects, the helical membrane may be disposed on an inner surface of the test strip 200, protected within the test strip 200.

The method may further include detecting an analyte in the biological sample. In some aspects, the increased interaction time provided by the extended helical flow path enhances binding between the analyte and binding reagents immobilized on the membrane or present in the conjugate. This enhanced binding may result in stronger signal generation at the detection pad 206, improving the detectability of the analyte.

In some aspects, detecting the analyte includes visual observation of a signal at the detection pad 206. The signal may be generated by accumulation of labeled conjugates at the test line, producing a visible color change or line. In other aspects, detecting the analyte includes fluorescence detection, where fluorescent labels on the conjugates are excited and detected using appropriate optical equipment. In some aspects, chemiluminescence detection may be employed, where chemical reactions produce light signals that indicate the presence of the analyte. Colorimetric readout may also be used, where color intensity correlates with analyte concentration. The detection method is not limited to these approaches and may include other suitable detection techniques.

In some aspects, the helical shape may include multiple turns arranged along a longitudinal axis of the test strip 200. The number of turns may be selected based on the desired flow path length, interaction time, and specific assay requirements. In some aspects, the helix may include two, three, four, five, or more turns. The pitch of the helix, defined as the distance between successive turns, may be uniform or may vary along the length of the helix to provide different flow characteristics in different regions. Aspects of the present disclosure are intended to include or otherwise cover any number of turns in the helix, without deviating from the scope of the present disclosure.

In some aspects, the extended path provided by the helical configuration may offer several functional benefits. In some aspects, the longer flow path may allow for more complete binding reactions between analytes and binding reagents, which may improve sensitivity and detection limits. In some aspects, the extended interaction time may enable detection of analytes present at lower concentrations that might not be reliably detected with shorter flow paths. The helical configuration may also provide more uniform flow distribution across the membrane, potentially improving assay reproducibility.

In some aspects, the biological sample may flow continuously from an inner turn toward an outer turn of the helical membrane, with the sample application point located near the center and the absorption occurring at the periphery. In some aspects, the flow direction may be reversed, with the biological sample applied at an outer turn and flowing inward toward the center. The flow direction may be selected based on the specific device design and application requirements.

In some aspects, the method of extending the sample flow path through helical arrangement of the membrane may provide a practical approach to increase interaction time and improve assay performance while maintaining a compact, portable device format suitable for point-of-care diagnostic applications. The geometric transformation from linear to helical configuration may represent a solution that enables extended flow paths without the corresponding increase in the test strip 200 footprint that may be required in conventional linear designs.

The helical configuration illustrated in FIG. 8 demonstrates the principle of using helical geometry to extend flow path length within a compact test strip 200. A straightforward implementation that can be readily manufactured may be provided by this configuration, and significant advantages in terms of increased interaction time and improved assay sensitivity may be offered while test strip 200 compactness may be maintained.

Referring to FIG. 8A, a variation of FIG. 8 is illustrated, showing a variable-pitch helical configuration. In this aspect, a helical strip having a variable pitch may be provided, whereby the spacing between successive turns of the helix may not be uniform. Some segments of the helix may be wound tightly with turns close together, while other segments may be more stretched out.

In some aspects, the test strip 200 may include the sample pad 202, the conjugate pad <not shown>, the detection pad 206, and the absorption pad 208 disposed on the surface of the test strip 200. Regions where the strip may be nearly horizontal versus steeply vertical relative to the central axis may be created by designing sections of the helix with different pitch angles. The biological sample flow may encounter these differing geometries as the biological sample travels through the helical membrane. A tight-pitch segment may be placed where a delay or longer interaction may be desired, and a loose-pitch segment may be placed where faster flow-through may be acceptable. The variable pitch may be achieved by changing the winding angle during manufacturing or by mounting the membrane on a support that has variable thread spacing.

In some aspects, built-in flow control zones may be provided by this configuration. Sections with a flatter or tighter pitch may effectively make the biological sample travel a longer horizontal distance per vertical rise. The capillary advance may be slowed by this configuration, particularly if the test strip 200 may be oriented so gravity opposes flow in those sections, whereby a time-delay element may be provided. In some aspects, steep pitch sections may allow the biological sample to climb quickly. By alternating these sections, flow may be paused at critical points, such as around the detection pad 206 to allow more binding time, and then may be accelerated afterwards. This approach may require no additional materials, as geometry alone may serve as the flow modulator.

In some aspects, gaps for inserting functional components may be provided by the varying pitch. A widened gap between turns may house a thicker reagent pad, a signal amplification zone, or a sensor, which might not fit if the helix were uniformly tight. Once the biological sample reaches that segment, interaction with the inserted component may occur, such as dissolving a delay barrier or mixing with an additional reagent. After passing the segment, the helix may tighten again whereby wicking may continue. This design flexibility may be useful for multi-step assays that require different materials or pads along the flow path.

In some aspects, the variable pitch helix may be designed such that the biological sample may experience a gradual transition in flow conditions. The pitch may progressively tighten to gently slow the flow over time. This may be used to create a gradient of incubation times, where an early part of the helix may move the biological sample fast, limiting background binding, and a later part may move the biological sample slow, maximizing signal at the detection pad 206. Such dynamic flow profiling may improve assay performance without external intervention.

In some aspects, the changing pitch may also serve as a visual indicator of different assay stages. Manufacturing and quality control may be aided by this feature, ensuring that specific segments may be properly positioned. Additionally, if an electronic reader may be used, different sections may be easily identified by their known spacing, and different reading modes may be applied. Overall, a programmable aspect to strip design through geometry may be added by variable-pitch helices.

Referring to FIG. 8B, a variation of FIG. 8 is illustrated, showing an interlaced dual-helix configuration. In this aspect, two helical lateral flow assay strips may be twisted around each other in a double-helix arrangement, reminiscent of a DNA double helix structure. The two strips may run in parallel but may physically intertwine, periodically coming into contact at crossover points.

In some aspects, each helix may carry a different reagent or may serve a different function. For instance, one helix might contain the primary flow with biological sample and detection antibodies, while the second helix may carry a secondary reagent such as a buffer or enzyme that merges at certain points. The strips may be arranged on a common support structure or simply wound together. Fluid transfer may be engineered at contact points by porous bridges or shared pads. An interactive multi-lane spiral may be created by the interlaced design, where two flows may communicate at specific junctures.

In some aspects, the test strip 200 may include the sample pad 202, the conjugate pad <not shown>, the detection pad 206, and the absorption pad 208 disposed on the surface of the test strip 200. These components may be distributed across one or both of the interlaced helices depending on the specific assay configuration.

In some aspects, sequential reagent introduction may be enabled by having two flows that meet at designed points, whereby this double-helix may perform multi-step assays automatically. For example, the biological sample may be delivered by the primary helix and may be allowed to bind at a test line <not shown> present on the detection pad 206, and a washing buffer or signal amplification reagent may be carried by the secondary helix that joins in later. At a contact junction, the second channel's fluid may wick into the first channel's membrane, whereby a timed introduction of a new reagent may be achieved. Complex protocols such as washes or amplifications may be enabled to occur in one test strip 200 by this crossover mechanism, which may be normally challenging in standard lateral flow assays.

In some aspects, dual analyte detection with interaction may be provided in multiplex scenarios, where each helix may target a different analyte. Both test lines <not shown> on the detection pad 206 may be in proximity, though on separate strips, due to the intertwined format, whereby a single reader may scan both simultaneously. Moreover, if desired, a reaction in one helix may be enabled to affect the other by the proximity. For instance, a strong positive in one channel might trigger a color change in the other as a built-in reference or to indicate a particular combined result. Possibilities for novel cross-signaling designs not possible with isolated strips may be opened by this interlacing.

In some aspects, mutual structural support may be provided by twisting two helices together, whereby the assembly may be more rigid and may stand upright or maintain shape better than a single thin coil. This may be useful for handling and potentially for inserting into cartridges. The braided structure might also resist kinking or collapsing, whereby reliability may be improved.

Increased surface area for binding may be provided when two helices touch, whereby their surface area in contact may be available for functionalization. Shared capture zones or high-capacity absorbent connections may be hosted by these contact sites. A three-dimensional network of membranes may be effectively created, leveraging more volume for the assay, somewhat akin to layered paper networks for signal enhancement. Sensitivity may be improved by trapping more analyte or reagents in the intersection regions.

Referring to FIG. 8C, a variation of FIG. 8 is illustrated, showing a multi-start helical configuration with parallel helix channels. In this aspect, multiple helical strips may be wound around the same axis in parallel, similar to a screw with multiple threads. Each helical channel may carry the biological sample through its own membrane path, but all may be fed by a common sample application point.

In some aspects, two or three helices may run side by side, each with its own set of reagents and test line, within one test strip 200 housing. Simultaneous lateral flow in each channel may be allowed by the co-parallel helixes. The overall structure may resemble a single coil with multiple intertwined strands. A built-in multi-channel lateral flow assay system in a compact spiral geometry may be essentially created by this multi-start helical design.

In some aspects, the test strip 200 may include the sample pad 202, the detection pad 206, and the absorption pad 208 disposed on the surface of the test strip 200. These components may be distributed across multiple helical channels, with each channel having its own detection pad 206 and absorption pad 208, while sharing a common sample pad 202.

Simultaneous multiplex testing may be enabled by this configuration, where each helix may be functionalized to detect a different analyte. Parallel multiplexed detection of multiple biomarkers from one biological sample may be enabled, whereby diagnostic throughput and efficiency may be increased compared to a single-strip assay.

Shared sample and controls may be provided by the configuration. The biological sample may be distributed to all helical channels by a single sample pad 202, whereby each test may receive the same sample conditions may be ensured. A common control line or reference in one channel may serve all, whereby redundancy may be reduced.

A compact form factor may be provided by winding multiple channels together. Several test strips may be housed by the test strip 200 in the space of one. Portability and compactness for point-of-care use may be improved, whereby multi-test capability may be achieved without a large cassette.

Redundancy and reliability may be enhanced by the parallel helix channels. Duplicate tests of the same analyte or internal calibration may be performed by the parallel helix channels, whereby result reliability may be increased. If one channel fails or may be inconclusive, a result may still be yielded by another channel, whereby false negatives may be reduced.

In operation, referring to FIG. 8, the biological sample is applied to the sample pad 202, whereby capillary action is initiated and the sample begins to flow through the helical membrane. As the sample contacts the conjugate pad, conjugate reagents are mobilized and mixed with the sample. The biological sample then flows along the helix of the helical membrane through multiple turns sequentially, traversing a significantly extended path compared to a conventional linear configuration. This extended flow path is achieved by the three-dimensional helical geometry, whereby the sample travels a distance multiple times longer than would be possible in a linear strip of equivalent footprint. As the sample progresses through each helical turn, continuous interaction with binding reagents immobilized on the membrane surface is maintained, whereby the residence time and interaction duration are substantially increased. Enhanced binding between analytes in the sample and the binding reagents is facilitated by this prolonged interaction time, potentially improving capture efficiency and signal generation. The sample eventually reaches the detection pad 206, where analyte-conjugate complexes are captured at the test line, producing a detectable signal. Excess sample is then absorbed by the absorption pad 208, whereby continuous unidirectional flow is maintained throughout the assay. The implementation of helical geometry thus decouples flow path length from test strip 200 footprint, enabling extended interaction time and improved sensitivity while maintaining a compact, portable form factor suitable for point-of-care applications.

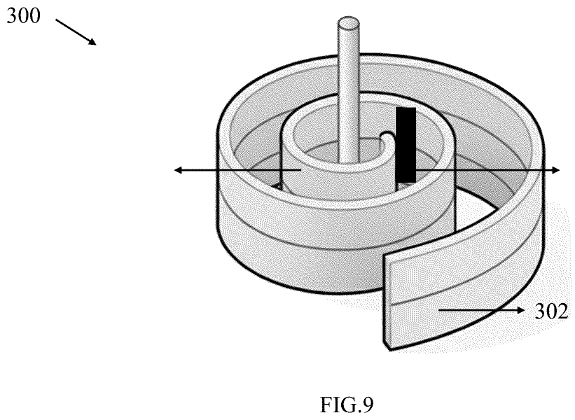

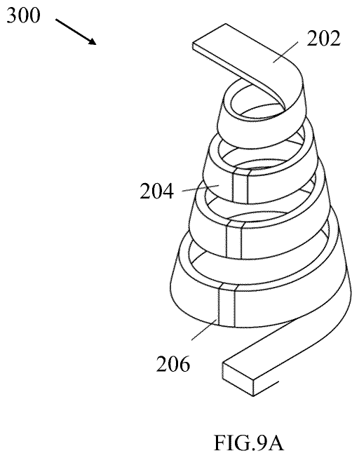

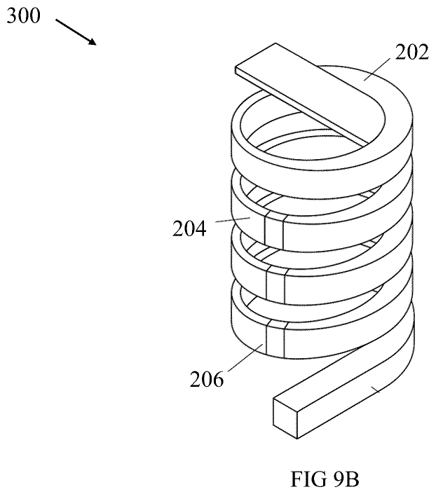

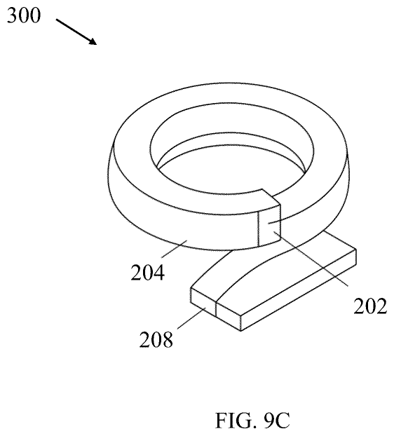

Referring to FIG. 9, a lateral flow assay test strip 300 having a nested coaxial helices configuration is illustrated. In this aspect, nested helical strips may be provided, where one smaller-diameter helix may sit inside a larger-diameter helix. The two helices may be concentric but not physically touching, and may be separated by a small gap or a permeable divider. The helices may be arranged coaxially like a spiral within a spiral, each handling a separate flow.

In some aspects, the outer helix may serve as the main test for the analyte, while the inner helix may carry a control or a second analyte test. Alternatively, one helix may serve as a pretreatment channel for filtering or mixing the biological sample with conjugates, whose output may then feed into the second helix for detection. Multiple functions may be kept co-located in one cylindrical volume without intertwining by the nested design.

In some aspects, the test strip 300 may include the sample pad 202, the detection pad 206, and the absorption pad 208 disposed on the surface of the test strip 300. These components may be distributed across the nested helices, with each helix having its own set of components or sharing certain elements depending on the specific assay configuration.

Multiplexing in minimal space may be enabled by the nested helices, allowing multiple channels and thus multiple tests in one test strip 300. The footprint may remain very small because one coil may be inside the other. An efficient way to pack two or more lateral flow tests for different targets into a single unit may be provided, potentially sharing the same sample input. The three-dimensional space may effectively utilize through a coaxial arrangement, with one helix occupying the outer ring and another occupying the inner core of the test strip 300.

In some aspects, sequential or hierarchical testing may be enabled by the nested setup. For example, a broad, high-sensitivity screening test may be performed by the outer helix. The same biological sample, perhaps drawn in after a delay or via diffusion, may then be used by the inner helix to perform a confirmatory test or a different assay that only runs if the outer may be positive. This could all occur in one test strip 300 automatically. One helix may be fed into the other if needed by a small connecting channel, whereby the proximity may be utilized.

Environmental isolation between channels may be provided because the helices may be separate. One channel's chemistry may be kept isolated until a designed junction. This may allow, for instance, the inner helix to contain reagents that would interfere with the outer helix's assay if mixed too early. Only when the flows reach a certain point, perhaps at the end or via a one-way valve, may they combine. Cross-talk between tests during most of the assay may be prevented by this isolation, ensuring specificity in multiplexed measurements.

Enhanced signal or control may be provided by dedicating the inner helix to a built-in control or calibration mechanism. For instance, a known analyte or fluorescence reference that runs concurrently to verify test operation may be carried by the inner helix. Being nested, the inner helix may experience the same conditions such as temperature and sample timing as the outer helix, making the inner helix a robust control. Additionally, nested designs could be extended to more than two layers, such as a triple-nested spiral, though alignment and manufacturing complexity would increase

The nested coaxial helices configuration illustrated in FIG. 9 may demonstrate an approach to multiplexing and hierarchical testing within a compact three-dimensional structure. Multiple independent flow paths may be provided within a single cylindrical volume by this configuration, whereby complex multi-analyte or multi-stage assays may be performed while maintaining a minimal test strip 300 footprint suitable for point-of-care applications.

Referring to FIG. 9A, a variation of FIG. 9 is illustrated, showing a conical helical configuration. In this aspect, the lateral flow strip may be formed into a conical helical shape, whereby the spiral's diameter may gradually change. The strip may be wide at the top and narrow at the bottom, like a cone spring. The test strip 300 may wind upward around a cone rather than a cylinder. This geometry may mean that one end of the helix has a larger radius and spacing between coils, and the other end may be tighter. The biological sample could be introduced at the wider end, allowing a larger initial volume spread, which may then funnel down to the narrower end as the biological sample travels. The conical helix may be oriented vertically or horizontally depending on use. If vertical with the wide end up, gravity and capillarity may interplay to influence flow speed along the changing radius.

In some aspects, the test strip 300 may include the sample pad 202, a conjugate pad <not shown>, the detection pad 206, and the absorption pad 208 disposed on the surface of the test strip 300. These components may be positioned along the conical helix to take advantage of the varying diameter and flow characteristics at different locations.

Controlled flow dynamics may be provided by the varying cross-sectional areas and capillary pressure along the conical spiral, whereby flow rate gradients may be created. Fluid may move more slowly in the wider sections and may speed up in narrower sections, or vice versa, helping to time the assay steps. Reaction incubation at certain regions could be improved by this built-in flow control. For example, slower flow at the test line may be achieved if the detection pad 206 may be placed in a wide segment, then faster wicking to the absorption pad 208 may occur in the narrower sections.

A sample concentration effect may be provided by starting the assay at a broad section and ending in a narrow section, whereby the biological sample and reagents may be funneled, potentially concentrating the analyte as the biological sample reaches the detection zone. Efficient uptake may be helped by a larger surface area at the sample application end, while the flow may be focused by a tighter end, potentially boosting sensitivity at the test line where the detection pad 206 may be positioned.

Enhanced capillary action may be provided if the conical shape may be oriented appropriately, whereby gravity may be leveraged. For instance, the capillary flow might be naturally slowed by an upward narrowing helix working against gravity as the diameter shrinks, providing more dwell time for binding. Conversely, flow may be accelerated by a downward narrowing helix. A new layer of assay optimization without additional parts may be added by this geometry-tuned flow control.

Ease of manufacturing alignment may be provided by the conical form, whereby a natural alignment for assembly may be created. Components like pads or electronic readers may be designed to slide over the cone. Insertion of a conical spiral into funnel-shaped sample collection test strip 300 or cartridges may also be easier, whereby user experience may be improved.

The conical helical configuration illustrated in FIG. 9A demonstrates how geometric variation in helix diameter may be utilized to control flow dynamics and enhance assay performance. Flow rate gradients and sample concentration effects may be provided by the tapered geometry, whereby sensitivity and timing control may be improved while maintaining a compact form factor.

Referring to FIG. 9B, a variation of FIG. 9 is illustrated, showing a folded helical configuration. In this aspect, folding or bending of the helical strip may be performed to create a segmented structure. A long helical coil may be bent back onto itself, or two helical sections may be stacked one above the other. For example, a helical strip may run upward, then at a midpoint the strip or support may be folded so that the remaining length of the strip may continue as a second helix adjacent to the first, like two springs side by side connected by a turn-around. Another approach may be a stacked spiral, where one helix may sit on top of another in layers, with perhaps a fluid connection or via gravity drip between layers. The folding may be facilitated by a flexible backing that allows the coil to bend without crimping the flow path. Essentially, a lateral flow assay that may not be a single continuous spiral but multiple spiral segments in one test strip 300 may be created by this configuration.

In some aspects, the test strip 300 may include the sample pad 202, the conjugate pad <not shown>, the detection pad 206, and the absorption pad 208 disposed on the surface of the test strip 300. These components may be distributed across the folded helical segments depending on the specific assay configuration.

An ultra-long path in compact form may be provided by folding a helix back onto itself, whereby the path length may be effectively doubled without extending the test strip 300's footprint. This may be useful for assays requiring extended run lengths for higher sensitivity or multiple reaction stages. By stacking helix segments, an extremely long serpentine flow in a volume-efficient package may be created. An alternative to the toroidal ring may be provided, whereby instead of looping 360°, the strip may fold 180° and may continue in parallel.

Multiple stages in one test strip 300 may be enabled by the folded helix, whereby the assay may be separated into distinct stages. For instance, biological sample introduction and conjugate mixing might be handled by the first helical segment, then detection and absorbance may be handled by the second segment after the strip folds. A small delay or barrier may be placed at the fold junction to ensure the first stage completes before the second begins. This segmentation may be akin to performing two connected lateral flow assays sequentially. Performance could be improved by optimizing each stage's environment, whereby the first helix could be treated to maximize binding of analyte to detector particles, and the second may be optimized for capturing at test lines.

In some aspects, versatile orientation may be provided by the folded helix, whereby the test strip 300 may be designed to unfold or deploy when needed. For example, the coil could be stored folded, minimizing size for packaging, and the test strip 300 might be unfolded or extended by the user before running the test, increasing spacing for clearer reading of results. Even if kept folded during operation, the adjacent coil sections may be oriented to suit the reader optics. For example, one section's test line may face one direction and the other section may face another, whereby both may be captured by a single camera by a mirror or dual view. Flexibility in integrating with various reader designs may be provided by this configuration.

In some aspects, spatial multiplexing or repeat testing may be enabled if each folded segment may be treated as a separate domain, whereby a folded helical lateral flow assay could run replicates or distinct tests on each segment from the same biological sample. For instance, the biological sample may split at the fork and may travel into two helix sections, one folded over the other. Each segment may have its own test line for different targets. This may be similar to multi-start configurations, but the segments need not be identical in geometry. Different lengths or pitches tailored to each analyte's needs could be provided by the segments. A fluid splitter may be incorporated at the fold junction to direct biological sample into multiple paths.

The folded helical configuration illustrated in FIG. 9B may demonstrate an approach to extending flow path length through folding and segmentation. An ultra-long serpentine flow path within a compact volume may be provided by this configuration, whereby multiple assay stages or multiplexed testing may be performed while maintaining test strip 300 compactness suitable for point-of-care applications.

Referring to FIG. 9C, a variation of FIG. 9 is illustrated, showing a toroidal helical configuration. In this aspect, the lateral flow strip may be wrapped into a toroidal or donut-shaped helix. The test strip 300 may coil around in a circle, forming a closed loop or ring of spirals. In practice, multiple turns around a ring-shaped support could be spiraled by the helix, with the ends of the strip meeting near each other, though not fluidly connected, to maintain a start and end. The result may be a lateral flow path that curves in three dimensions and returns near its starting point, resembling a slinky bent into a circle. The test and control line regions could be distributed around the ring. Biological sample might be added by a user at one point on the ring, and the fluid may travel around the torus to an absorbent pad on the opposite side.

In some aspects, the test strip 300 may include the sample pad 202, a conjugate pad <not shown>, the detection pad 206, and the absorption pad 208 disposed on the surface of the test strip 300. These components may be distributed around the toroidal helix depending on the specific assay configuration.

An ultra-compact configuration may be provided by curving the helix back onto itself in a ring, whereby the test strip 300 footprint may become very small and space-efficient. Round casings, like a watch battery size, or even wearable formats may be fitted by a toroidal helix. Lateral flow assays that may be integrated into compact test strip 300s or cartridges where a linear strip would not fit could be enabled by this configuration.

Extended path length may be provided by the spiral looping around the torus multiple times, achieving a very long flow path in a confined space. More time and distance for reactions may be allowed by this extended path, potentially increasing sensitivity and enabling multiple test zones spaced along the loop. The detection pad 206 having control lines <not shown> may be positioned at various points around the toroidal path to take advantage of the extended interaction time.

In some aspects, 360° detection access may be allowed by the ring shape, whereby detectors, whether optical or electromagnetic, may scan the test strip 300 from all sides. For example, rotation around the ring or viewing of the entire circumference could be performed by an optical reader. Electronic reading may be facilitated whereby the test strip 300 could be placed in a donut-shaped reader where sensors may surround the test strip 300 to detect signals from any angle. Signal detection may be improved by this omnidirectional access, and more sophisticated reading mechanisms may be enabled.

Potential for repeated or circular flow may be provided by the toroidal format. Although a true continuous loop would require external pumping, fluid recirculation in a closed circuit may be conceptually allowed by the toroidal format. With additional microfluidic valves or a slight gap, multiple passes over the test line could be performed by the fluid, enhancing signal, before final absorption. Even without active recirculation, sequential biological sample addition at intervals around the ring for repeated measurements or quality control checks in one test strip 300 might be permitted by the geometry.

The toroidal helical configuration illustrated in FIG. 9C demonstrates an approach to achieving maximum compactness while providing extended flow path length. An ultra-compact, space-efficient test strip 300 with omnidirectional detection access may be provided by this configuration, whereby sensitivity may be enhanced and novel reading mechanisms may be implemented while maintaining a minimal footprint suitable for wearable or highly portable point-of-care applications.

In operation, referring to FIG. 9, the biological sample is applied to the sample pad 202, whereby the sample is distributed to both the outer helix and the inner helix of the nested coaxial configuration. Each helix provides an independent flow path, whereby multiple tests or sequential processing stages can be performed simultaneously within a single compact test strip 300. The outer helix may perform a primary assay, where the sample flows through multiple helical turns, interacting with binding reagents over an extended path length to maximize sensitivity. Concurrently or sequentially, the inner helix may perform a control test, a confirmatory assay, or a pretreatment step such as filtering or reagent mixing. The extended flow path provided by each nested helix increases the interaction time between the sample and binding reagents, whereby binding efficiency and detection sensitivity are enhanced. The coaxial arrangement utilizes three-dimensional space efficiently, whereby two or more independent flow channels are accommodated within a minimal footprint. Environmental isolation between the helices is maintained until designed junction points, whereby cross-talk between different assays is prevented and specificity is ensured. Detection is performed at the detection pad 206 on each helix, and excess sample is absorbed by the absorption pad 208. The nested coaxial design thus enables multiplexed or hierarchical testing with extended flow paths in a compact cylindrical volume, whereby complex multi-analyte or multi-stage assays can be performed with improved sensitivity while maintaining test strip 300 portability for point-of-care applications.

In some embodiments, the detection zone geometry within a helical lateral flow assay may be designed by providing a three-dimensional test line structure instead of a traditional flat test line. The test line could wrap around the circumference of the helix, covering all sides of the strip rather than just a single line on the flat face, whereby a band of capture antibody that intercepts flow from all directions may be created. Alternatively, a small cylindrical sponge or a porous three-dimensional pad may be inserted at the test line location on the helix, effectively providing a volumetric region where the target is captured. The helical support might also include ridges or microstructures that make the test line zigzag through the thickness of the membrane, whereby contact area is increased. The helix's three-dimensional shape my leverage to move beyond a two-dimensional line and create a volumetric capture zone for the analyte. Higher binding capacity is provided by a three-dimensional test line, whereby more surface area and volume to capture analytes are presented. Significantly more binding sites may be made available by having the test antibody coated on an all-around band or within a porous structure than would be available with a thin line on a flat membrane, whereby the dynamic range and sensitivity of the test can be increased as more of the target can be trapped and displayed. Improved signal visibility may be provided whereby a test line wrapped 360° around a helix or made thicker can yield a stronger visible signal. As gold nanoparticles or colored labels accumulate in a thicker band, the color intensity builds up, making faint positives easier to see, whereby a more robust readout that could enable semi-quantitative assessment by the naked eye or easier thresholding by a reader is provided. Fast uptake and wicking are enabled whereby a porous three-dimensional capture pad can quickly wick fluid from all sides, potentially acting as a capillary sink that draws sample through the test zone efficiently. Bottlenecks at the test line are prevented, whereby even as the pad holds fluid longer for binding, excess sample can be absorbed to speed up overall run time, and sensitivity gain without slowing the assay too much is achieved. Layered or multi-analyte capture may enable whereby the three-dimensional space may be used to layer multiple reagents. For example, one layer with primary capture antibody and beneath a second layer with another capture or a secondary reaction such as a color amplification enzyme might be provided by a three-dimensional pad. The sample passes through successive micro-environments by this vertical layering. This three-dimensional structure may conveniently held in place by the helix form factor, whereby some microfluidic three-dimensional architecture may essentially be brought into the paper strip, all within a single helical coil.

In some embodiments, the helical lateral flow assay may be augmented with active flow control mechanisms. Rather than relying solely on capillary action, small microfluidic actuators such as miniature pumps, valves, or even external forces like centrifugation may be incorporated by the test strip 300 to drive and control the fluid. In some aspects, the design might include a tiny battery-powered pump at the absorbent end to pull the sample through at a controlled rate, or periodically stop and start the flow. Alternatively, the helix could be mounted in a cartridge that spins, whereby centrifugal force may be used to actively regulate flow, achieving precise timing of when fluid moves through each coil. Micro valves, such as hydrophobic patches that maybe opened by an external trigger such as pressing a button or applying light or heat, may also be placed along the helix to reroute or delay flow on demand. Integration of lateral flow with lab-on-chip fluidics may be effectively created by the active-flow helix, whereby the sample wicks into the helix, but then the journey is modulated by active control elements. Finer control over kinetics may be provided whereby active pumping or valving allows the assay to optimize reaction times far beyond passive capillary limits. For instance, the flow may be slowed to a crawl by a pump when the sample reaches the test line, then sped up after binding is done, maximizing sensitivity and then minimizing total assay time. Better performance and reproducibility with less variability due to environmental factors can be yielded by this precise control. Multi-step protocol execution may enable whereby with valves and controlled flow, washing steps, reagent introductions, and even multistage reactions may be performed more reliably by the test strip 300. A helix could be designed that first pumps the sample through, then at a set time opens a valve to allow a stored wash buffer to flush the test line, and finally directs a substrate solution to flow, mimicking an ELISA workflow in a self-contained format. Such complexity would normally require a laboratory setup, but the test strip 300 itself orchestrates the process, whereby point-of-care testing is brought closer to lab analyzer capabilities. Rapid and automated results may be provided whereby active flow can significantly reduce the assay time by forcing fluids through faster when extended incubation is not needed, and by parallelizing steps. For example, a segment of the helix may be pre-filled with buffer and held by a closed valve, whereby while the sample may be incubated in one coil, the next coil's buffer can be ready, then quickly released. A faster turnaround and the possibility of automation may provide as the net result, whereby a small desktop test strip 300 that hosts the helical strip, controls the flow, and outputs a result without user intervention beyond loading the sample could be envisioned. Handling of viscous or challenging samples may be improved whereby in some cases such as whole blood or saliva, capillary flow may be erratic or slow. Consistent flow even with viscous fluids or when the strip pores start to clog may be ensured by an active pump in the helical lateral flow assay, whereby reliability is improved and the range of samples that can be tested is broadened. Additionally, larger sample volumes may be processed through the strip to reach lower detection limits by actively pulling fluid, whereby the test strip 300 could, for example, cycle more sample through the test region in a controlled manner.

### ADVANTAGES OF THE INVENTION

Extended Flow Path in Compact Footprint: A significantly longer flow path within the same or smaller spatial dimensions may be achieved by arranging the membrane in a helical configuration. The flow path is transformed from a two-dimensional linear path into a three-dimensional winding path by the helical geometry, whereby the flow path length is decoupled from the device footprint. For example, a flow path five times longer than a single linear strip of the same width may be provided by a helical membrane with five turns, yet a footprint determined primarily by the diameter of the helix rather than the total path length is occupied. Extended interaction time is enabled without the corresponding increase in the size of the test strip that would be required in conventional linear designs by this geometric transformation.

Enhanced Interaction Time and Binding Efficiency: Substantially increased residence time of the biological sample within the membrane is provided by the extended helical flow path. As the sample travels through multiple turns of the helix, continuous interaction with binding reagents immobilized on the membrane surface is maintained over a significantly longer duration compared to linear configurations. More extensive binding reactions between analytes and binding reagents are allowed by this prolonged contact time, whereby capture efficiency at detection pad 206 is improved and signal generation is enhanced. Detection of analytes present at lower concentrations that might not be reliably detected with shorter flow paths may be enabled by the increased interaction time, whereby assay sensitivity is substantially improved.

Geometry-Based Flow Control: Flow velocity and residence time can be modulated through geometric design alone by the helical configuration, without requiring additional materials or components. The three-dimensional helical path, combined with the increased surface area of the membrane, creates capillary forces that naturally moderate the flow rate. Flow characteristics in different regions of the assay can be tailored by varying the pitch of the helix, whereby tighter pitch segments provide longer interaction times and looser pitch segments allow faster flow-through. Built-in flow control zones that optimize reaction kinetics at critical points such as detection pad 206 are created by this geometry-tuned approach, whereby assay performance is improved without external intervention.

Maintained Portability for Point-of-Care Applications: The test strip is allowed to remain small and portable despite the extended flow path by the three-dimensional helical arrangement, making the configuration suitable for point-of-care applications. The benefits of extended interaction time and improved sensitivity are achieved while the compact form factor essential for field settings and point-of-care use is preserved. Ease of handling, storage, and deployment in resource-limited settings is facilitated by this combination of enhanced performance and maintained compactness, whereby the competing requirements of assay sensitivity and device portability are simultaneously addressed.

Multiplexing and Hierarchical Testing Capabilities: Multiple independent flow channels within a single compact device are enabled by configurations such as nested coaxial helices and multi-start helical designs. Simultaneous detection of multiple biomarkers from one sample is allowed by these configurations, whereby diagnostic throughput and efficiency are increased. Sequential or hierarchical testing can be performed whereby screening tests are followed by confirmatory assays within the same device, all occurring automatically. Environmental isolation between channels is maintained until designed junction points are designed, whereby cross-talk between different assays is prevented and specificity in multiplexed measurements is ensured.

Three-Dimensional Volumetric Capture Zones: Higher binding capacity and improved signal visibility are provided by incorporating three-dimensional test line structures within the helical configuration. Significantly more binding sites are made available by wrapping the test line around the circumference of the helix or inserting porous three-dimensional capture pads than would be available with traditional flat test lines. The dynamic range and sensitivity of the test are increased as more target analytes can be trapped and displayed. Stronger visible signals are yielded as labeled particles accumulate in thicker volumetric bands, whereby faint positives are made easier to detect and semi-quantitative assessment is enabled. The three-dimensional helical geometry thus provides both extended flow paths and enhanced capture zones, whereby multiple aspects of assay performance are simultaneously improved.

A number of implementations have been described. Nevertheless, it will be understood that various modifications may be made without departing from the spirit and scope of the disclosure. Accordingly, other implementations are within the scope of the following claims.

We claim

1. A method of extending the path of a biological sample flow in a lateral flow assay on a test strip comprising a membrane, said method comprising arranging the membrane in a helical shape in a test strip.

2. The method of claim 1, wherein the test strip further comprises a sample pad and a detection pad disposed on the surface of the test strip.

3. The method of claim 1, wherein the helical membrane is disposed completely or partially on the inner or outer surface of the test strip.

4. The method of claim 1, wherein the biological sample flows along the helix of the helical membrane.

5. The method of claim 1, wherein the membrane is configured to facilitate biological sample flow in a direction substantially perpendicular to a central axis of the helical shape in the test strip.

6. The method of claim 1, wherein the detection pad is disposed on the test strip, such that the biological sample flows into the detection pad in a direction substantially perpendicular to a central axis of the helical membrane.

7. The method of claim 1, wherein the extended path increases interaction time between the biological sample and binding reagents on the membrane.

8. The method of claim 1, wherein the biological sample flows through multiple turns of the helix sequentially.

9. The method of claim 1, wherein the biological sample flows in a direction substantially perpendicular to a central axis of the membrane.

10. The method of claim 1, further comprising detecting an analyte in the biological sample, wherein the increased interaction time enhances binding between the analyte and binding reagents.

11. The method of claim 1, wherein the biological sample flows radially outward from the central axis.

12. The method of claim 1, wherein the biological sample flows radially inward toward the central axis.

13. The method of claim 1, wherein detecting the analyte comprises visual observation, fluorescence, chemiluminescence, or colorimetric readout.

14. The method of claim 1, wherein the helical configuration reduces flow velocity, thereby increasing residence time of the biological sample within the membrane.

15. A lateral flow assay on a test strip comprising a membrane disposed on a surface of the test strip in a helical shape, wherein the helical shape extends a path of a biological sample flow through the membrane.

16. The lateral flow assay of claim 15, wherein the helical shape increases interaction time between the biological sample and binding reagents on the membrane.

17. The lateral flow assay of claim 15, further comprising the detection pad disposed on the surface of the test strip.

18. The lateral flow assay of claim 15, wherein the membrane is configured to facilitate biological sample flow in a direction substantially perpendicular to a central axis of the helical shape.

20. The lateral flow assay of claim 15, wherein the biological sample is selected from urine, semen, sperm, blood, and saliva.

21. The lateral flow assay of claim 15, wherein the biological sample flows continuously from an inner turn toward an outer turn of the helical membrane.

22. The lateral flow assay of claim 15, wherein the helical shape comprises multiple turns arranged along a longitudinal axis of the test strip.
